(19) United States
(12) Patent Application Publication
(10) Pub. No.: US 2025/0367394 A1
(43) Pub. Date: Dec. 4, 2025

(54) TUBE SECURING DEVICE

(71) Applicant: Avent, Inc., Alpharetta, GA (US)

(72) Inventors: Emily BRAYMAN, Alpharetta, GA (US); Preston A. MOELLER, Alpharetta, GA (US); Travis W. KLYBER, Alpharetta, GA (US)

(21) Appl. No.: 18/854,357

(22) PCT Filed: Apr. 3, 2023
(86) PCT No.: PCT/US2023/017264

§ 371 Date: Oct. 4, 2024

Related U.S. Application Data
(60) Provisional application No. 63/327,400, filed on Apr. 5, 2022.

(51) Int. Cl. A61M 16/04; A61M 16/06

(52) U.S. Cl. CPC A61M 16/0497; A61M 16/0688; A61M 2209/088

(57) ABSTRACT

A tube securing device is provided that includes an upper clamp having an upper surface and a lower surface; a lower clamp having an upper surface and a lower surface; and a hinge located between the upper clamp and the lower clamp to define a tube insertion region. Further, a ratio of a length of the upper surface of the upper clamp to a length of the lower surface of the lower clamp ranges from about 0.95 to about 1.20. Without intending to be limited by any particular theory, the present inventors have found that such a ratio allows for the tube securing device to accommodate various tube sizes without restricting the flow of fluids while at the same time ensuring that the tubes do not become dislodged.

### RELATED APPLICATIONS

The present application claims priority to U.S. Provisional Patent Application No. 63/327,400, filed Apr. 5, 2022, the entire contents of which are incorporated herein by reference.

### FIELD OF THE INVENTION

The present invention relates generally to a tube securing device for securing a tube, such as a feeding tube, catheter, IV tube, etc., against a patient's skin to prevent the feeding tube from becoming dislodged.

### BACKGROUND

Tubes are used in various medical applications in order to deliver fluid or remove fluid from a patient. For example, feeding tubes, such as nasoenteric feeding tubes, are used to are used to provide nutritional support in critically ill patients. However, nasoenteric feeding tubes may easily become dislodged due to patient mental status, transfers, or positional changes. Research suggests that from about 40% to about 62% of nasoenteric feeding tubes become dislodged at some point during use. Tube dislodgement may lead to delayed enteral nutrition, increased risk of aspiration, or malposition of the feeding tube at the time of replacement, leading to increased health care costs and an increase in the amount of time spent replacing tubes. To further complicate matters, there are numerous sizes of nasoenteric feeding tubes on the market depending on the manufacturer, and the securing devices currently on the market used to prevent the dislodgement of nasoenteric feeding tubes are not compatible with all nasoenteric feeding tube sizes and brands and typically only work well to secure a nasoenteric feeding tube having a particular outer diameter. This leads to additional problems for healthcare providers in ensuring that nasoenteric feeding tubes are securely placed to provide optimum benefit to the patient. In other words, the standard clamp configurations that are currently available are designed to only accommodate one specific size of nasoenteric feeding tube.

Thus, a need exists for a tube securing device that can accommodate tubes having varying outer diameters in a secure manner to prevent dislodgement of the tubes during use.

### SUMMARY OF THE INVENTION

Objects and advantages of the invention will be set forth in part in the following description, or may be obvious from the description, or may be learned through practice of the invention.

In one particular embodiment, the present disclosure is directed to a tube securing device. The tube securing device includes an upper clamp having an upper surface and a lower surface; a lower clamp having an upper surface and a lower surface; and a hinge located between the upper clamp and the lower clamp to define a tube insertion region. Further, a ratio of a length of the upper surface of the upper clamp to a length of the lower surface of the lower clamp ranges from about 0.95 to about 1.20.

In another embodiment, the length of the upper surface of the upper clamp can range from about 1.25 millimeters to about 1.7 millimeters.

In still another embodiment, the length of the lower surface of the lower clamp can range from about 1.2 millimeters to about 1.6 millimeters.

In yet another embodiment, the upper surface of the upper clamp and the lower surface of the lower clamp can be slidably engaged when the upper clamp is depressed towards the lower clamp to secure a tube within the tube insertion region. Further, the upper surface of the upper clamp and the lower surface of the lower clamp can form an angle with each other ranging from about 0° to about 25° when slidably engaged in order to accommodate a large range of tube sizes.

In an additional embodiment, the tube securing device can include a protrusion extending from a base of the tube securing device adjacent the hinge.

In one more embodiment, the tube insertion region can have a radius, where a ratio of the radius of the tube insertion region to an outer diameter of a tube inserted into the tube insertion region can range from about 1:1 to about 1:3.

In another embodiment, at least one protrusion can extend from the lower surface of the upper clamp.

In still another embodiment, the upper surface of the lower clamp can include a first indentation at a first edge, a second indentation at a second edge, and a raised portion defined therebetween.

In yet another embodiment, the upper surface of the upper clamp and the lower surface of the upper clamp can define a trapezoidal shape where the upper surface and the lower surface meet.

In an additional embodiment, the tube securing device can include an intermediate layer having an upper surface and a lower surface, where the upper surface of the intermediate layer can be attached to a lower surface of a base of the tube securing device.

Additionally, the tube securing device can also include a nonwoven layer having an upper surface and a lower surface, where the upper surface of the nonwoven layer can be attached to the lower surface of the intermediate layer.

Further, the tube securing device can include an adhesive layer having an upper surface and a lower surface, where the upper surface of the adhesive layer can be attached to the lower surface of the nonwoven layer. Moreover, the lower surface of the adhesive layer can include an adhesive and can be attached to a removable release liner.

In one more embodiment, the tube securing device can secure a tube having a size of 6 French, 8 French, 10 French, 12 French, or 14 French. Further, the tube can be formed from silicone, polyurethane, polyvinyl chloride, or a combination thereof.

In another embodiment, the tube securing device can be configured to secure a feeding tube, such as a nasoenteric feeding tube.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a side perspective view of one embodiment of the tube securing device of the present disclosure;

FIG. 2 is a front perspective view of one embodiment of the tube securing device of the present disclosure;

FIG. 3 is a rear perspective view of one embodiment of the tube securing device of the present disclosure;

FIG. 4 is a rear view of one embodiment of the tube securing device of the present disclosure;

FIG. 5 is a front perspective view of another embodiment of the tube securing device of the present disclosure;

FIG. 6 is a side view of another embodiment of the tube securing device of the present disclosure;

FIG. 7 is a side view of the tube securing device of the present disclosure that has been secured about a feeding tube having a first outer diameter;

FIG. 8 is a side view of the tube securing device of the present disclosure that has been secured about a feeding tube having a second, larger outer diameter compared to the feeding tube in FIG. 7;

FIG. 9 is a front perspective view of the tube securing device of the present disclosure showing the various layers used to attach the tube securing device to a surface of a patient's skin; and

FIG. 10 illustrates the tube securing device in use to secure a nasoenteric feeding tube to a patient's skin after insertion of a portion of the nasoenteric feeding tube into a patient's nostril.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

Generally speaking, the present invention is directed to a tube securing device that includes two clamps (e.g., an upper clamp and a lower clamp) having surfaces that slide against one another to accommodate for various feeding tube sizes (e.g., feeding tubes available from Cardinal, Corflo, NeoMed (Avanos), and Medicina). The two clamp surfaces contact each other and slide against each other in order to accommodate the various tube sizes, where the length of each surface results in a particular ratio, as discussed in more detail below, that allows for the secure clamping of various tube sizes without overly restricting the flow of the tubes. Further, in some instances, the surfaces form an angle with each other of about 25° or less, and, in some embodiments, can be generally parallel with each other (e.g., they can form an angle with each other of about 0°).

In one particular embodiment, the tube securing device includes an upper clamp having an upper surface and a lower surface; a lower clamp having an upper surface and a lower surface; and a hinge located between the upper clamp and the lower clamp to define a tube insertion region. Further, a ratio of the length of the upper surface of the upper clamp to a length of the lower surface of the lower clamp ranges from about 0.95 to about 1.20. Without intending to be limited by any particular theory, the present inventors have found that such a ratio allows for the tube securing device to accommodate various feeding tube sizes without restricting the flow of nutrients through the feeding tubes while at the same time ensuring that the feeding tubes do not become dislodged.

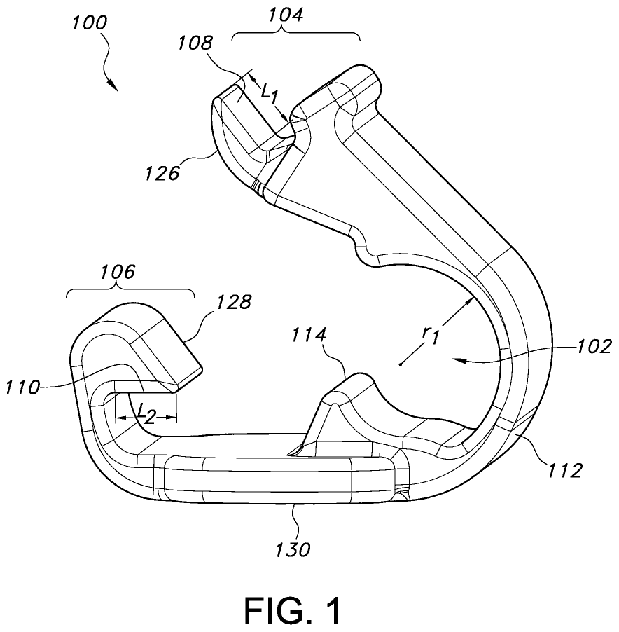

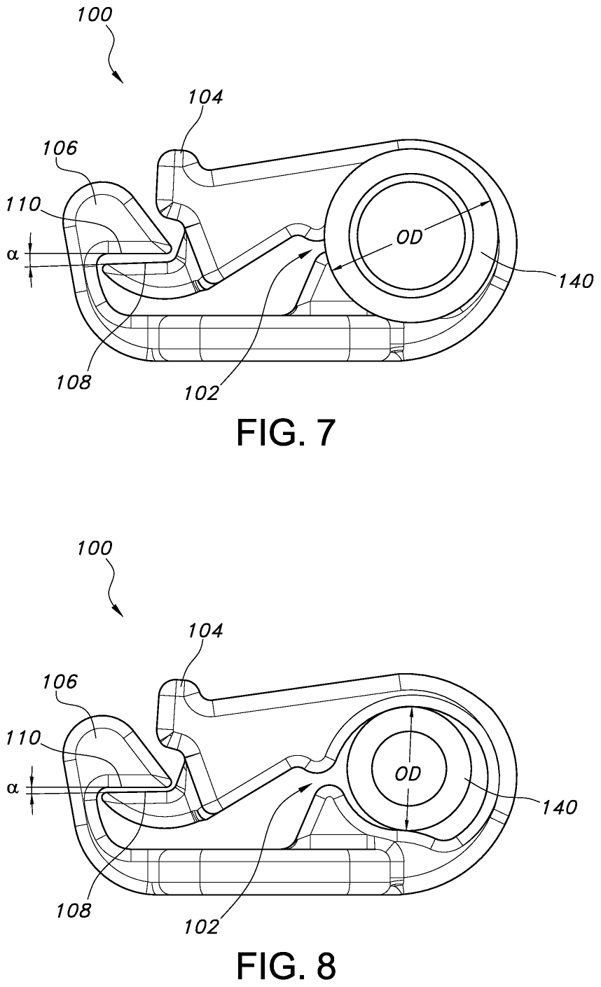

Referring now to the drawings, FIG. 1 is a side perspective view of one embodiment of the tube securing device 100 of the present disclosure. The tube securing device 100 includes an upper clamp 104, a lower clamp 106, and a hinge 112 located between the upper clamp 104 and the lower clamp 106 to define a tube insertion region 102. The tube insertion region 102 has a radius r₁ and is configured to receive a tube 140 (see FIGS. 7, 8, and 10). The radius r₁ can range from about 1.3 millimeters to about 3.3 millimeters, such as from about 1.4 millimeters to about 3.2 millimeters, such as from about 1.5 millimeters to about 3.1 millimeters to accommodate various tube sizes to secure and prevent dislodgement of the tubes while at the same time not overly restricting the flow through the tubes. Once a tube 140 is inserted into the tube insertion region 102, the upper clamp 104 and the lower clamp 106 are engaged at their respective upper surface 108 and lower surface 110 to secure the tube 140 in the tube insertion region 102 to prevent dislodgement of the tube 140. In addition, a protrusion 114 at the base 130 of the tube securing device 100 also serves to hold the tube 140 in place within the tube insertion region 102.

As shown, the upper surface 108 of the upper clamp 104 has a length L₁ and the lower surface 110 of the lower clamp 106 has a length L₂. The specific lengths and the ratio of the length L₁ of the upper surface 108 of the upper clamp 104 to the length L₂ of the lower surface 110 of the lower clamp 106 are specifically controlled so that the tube securing device 100 can expand and contract to accommodate various tube sizes while keeping the tube secure without overly restricting the flow. In this regard, the length L₁ of the upper surface 108 of the upper clamp 104 can range from about 1.25 millimeters to about 1.7 millimeters, such as from about 1.3 millimeters to about 1.6 millimeters, such as from about 1.35 millimeters to about 1.55 millimeters. Meanwhile, the length L₂ of the lower surface 110 of the lower clamp 106 can range from about 1.2 millimeters to about 1.6 millimeters, such as from about 1.25 millimeters to about 1.55 millimeters, such as from about 1.3 millimeters to about 1.5 millimeters. Further, the ratio of the length L₁ of the upper surface 108 of the upper clamp 104 to the length L₂ of the lower surface 110 of the lower clamp 106 can range from about 0.95 to 1.20, such as from about 0.96 to about 1.15, such as from about 0.97 to about 1.10.

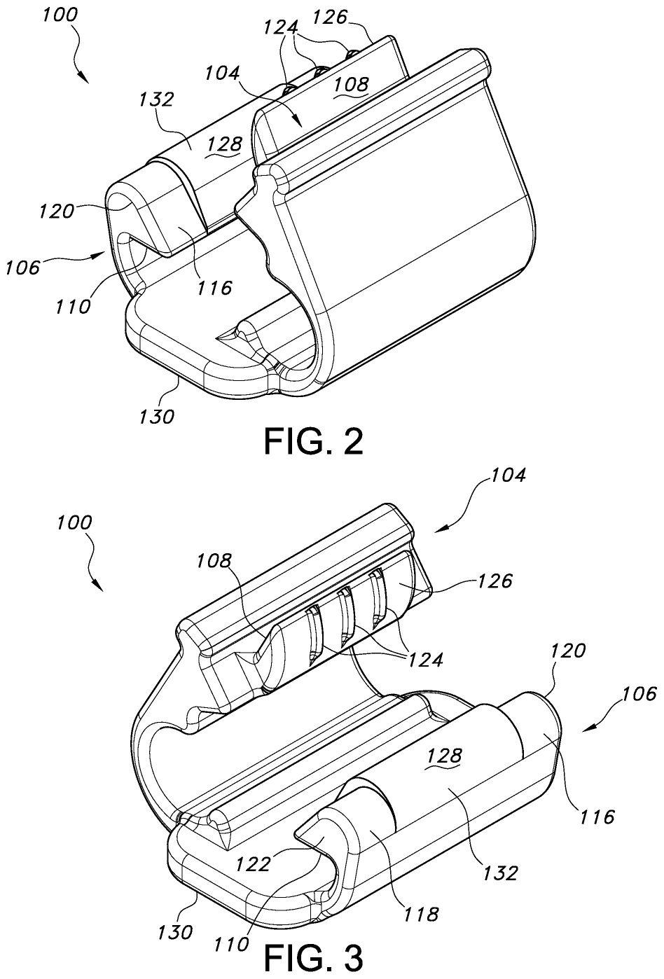

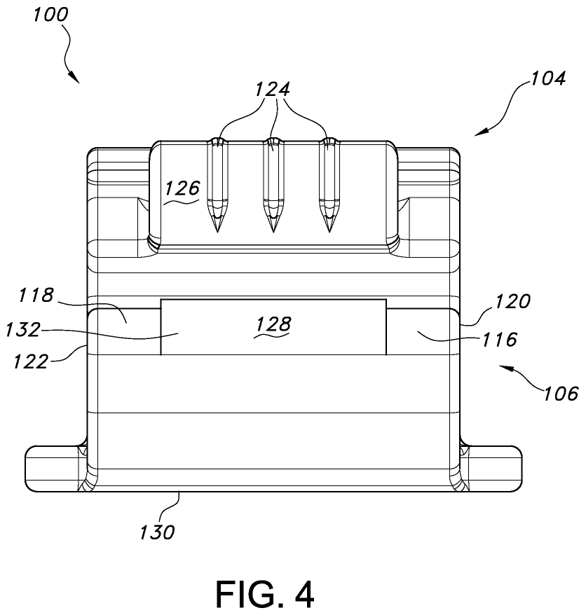

Referring now to FIGS. 2-4, other features that may be incorporated into the upper clamp 104 and the lower clamp 106 are shown, where such features reduce the friction between the two clamps when pinching or depressing the tube securing device 100 to engage the upper surface 108 of the upper clamp 104 with the lower surface 110 of the lower clamp 106 to secure a tube within the tube insertion region 102 upon flexing of the hinge 112 as the upper clamp 104 and the lower clamp 106 become slidably engaged. Specifically, the lower surface 126 of the upper clamp 104 may include one or more protrusions 124 or other forms of texturing, bumps, etc. to reduce surface friction as the lower surface 126 of the upper clamp 104 must move past the upper surface 128 of the lower clamp 106 in order to allow the upper surface 108 of the upper clamp 104 to come into contact with and slide against the lower surface 110 of the lower clamp 106.

In addition, the upper surface 128 of the lower clamp 106 can have a non-planar geometry to further aid in reducing the friction between the lower surface 126 of the upper clamp 104 and the upper surface 128 of the lower clamp 106 as the tube securing device 100 is depressed or pinched at the upper clamp 104 so that upper surface 108 of the upper clamp 104 can move past the upper surface 128 of the lower clamp 106 in order to allow the upper surface 108 of the upper clamp 104 to come into contact with and slide against the lower surface 110 of the lower clamp 106. For instance, the lower clamp 106 can include a first indentation 116 at a first edge 120, a second indentation 118 at a second edge 122, and a raised portion 132 defined therebetween to create multiple surface levels and help reduce friction as the upper clamp 104 is depressed or pinched to engage with the lower clamp 106.

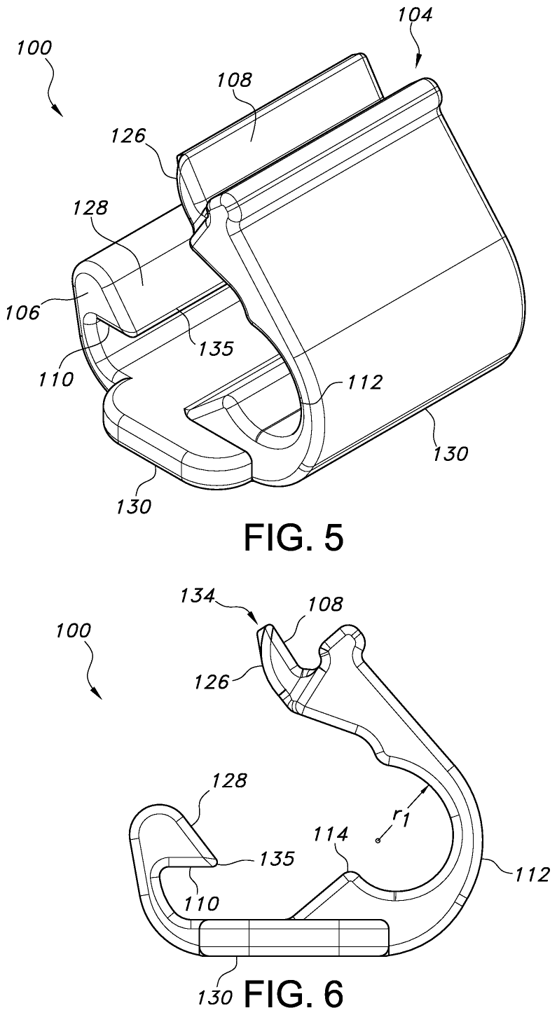

Referring now to FIGS. 5-6, another feature that may be included in the tube securing device 100 in order to reduce friction and improve the ease with which the upper clamp 104 slides against the lower clamp 106 is the geometry of the upper clamp 104. As shown in FIGS. 5-6, the upper surface 108 of the upper clamp 104 and the lower surface 126 of the upper clamp 104 can define a trapezoidal shape 134 where the upper surface 108 and the lower surface 126 meet, where the trapezoidal shape 134 can be the only portion of the upper clamp 104 that contacts a front edge 135 of the lower clamp 106 as the upper clamp 104 is being depressed or pinched so that the upper surface 108 of the upper clamp 104 can ultimately engage with the lower surface 110 of the lower clamp 106 to secure a tube in place effectively.

Next, FIG. 7-8 show the how a tube 140 is secured in the tube securing device 100 of the present disclosure. In particular, FIG. 7 is a side view of the tube securing device 100 of the present disclosure that has been secured about a feeding tube 140 having a first outer diameter OD, while FIG. 8 is a side view of the tube securing device 100 of the present disclosure that has been secured about a feeding tube 140 having a second, larger outer diameter OD compared to the feeding tube 140 in FIG. 7. Generally, the lower surface 110 of the lower clamp 106 contacts the upper surface 108 of the upper clamp 104 after the upper clamp 104 has been depressed or pinched to secure a tube 140 that has been inserted into the tube insertion region 102. Depending on the outer diameter OD of the tube 140, an angle a can be formed between the upper surface 108 of the upper clamp 104 and the lower surface 110 of the lower clamp 106. As shown in FIGS. 7 and 8, when the outer diameter OD of the tube 140 is larger, the angle a is smaller than when a tube 140 having a smaller outer diameter 140 is used (e.g., the upper surface 108 of the upper clamp 104 and the lower surface 110 of the lower clamp 106 form an angle of about 0° and are generally parallel). In any event, the upper surface 108 of the upper clamp 104 and the lower surface 110 of the lower clamp 106 can form an angle a with each other ranging from about 0° to about 25°, such as from about 0° to about 15°, such as from about 0° to about 7.5° when slidably engaged to secure a tube 140 in the tube insertion region 102 of the tube securing device 100.

In addition, the ratio of the radius r₁ to the outer diameter OD of the tube 140 can range from about 1:1 to about 1:3, such as from about 1:1.1 to about 1:2, such as from about 1:1.2 to about 1:1.5, where the present inventors have found that such a ratio ensures that the tube 140 is adequately secured within the tube securing device 100 without too much restriction of flow. For example, based on the ratio described above, the use of a force of greater than or equal to about 3 Newtons is required in order to pull the tube 140 through the tube securing device 100. Moreover, the flow rate through a tube 140 (e.g., a secured single lumen or large lumen of a dual lumen tube) secured within the tube securing device 100 is at least 70% of a baseline flow rate when tested per ISO 20695 Annex E when the ratios ranges described above are maintained.

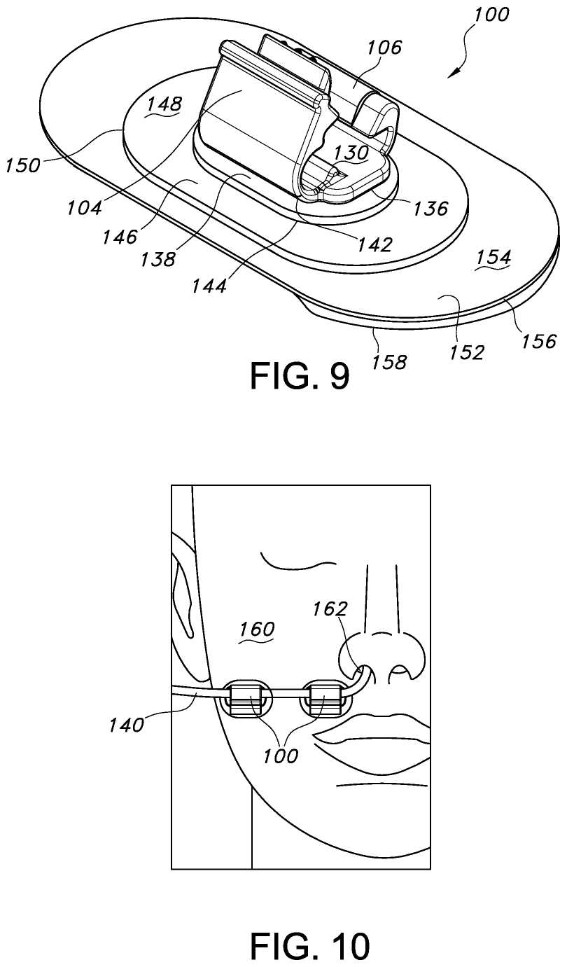

Turning now to FIG. 9, the various layers that can be used to attach the tube securing device 100 to a patient's skin 160 (e.g., the patient's face or cheek) are shown. For instance, the tube securing device can include an intermediate layer 138, which can, in some embodiments, be a foam layer. An upper surface 142 of the intermediate layer intermediate 138 can be attached to a lower surface 136 of the base 130 as shown. The intermediate layer 138 can provide additional support and cushioning to allow for optimal placement of the tube securing device 100 and also flexes and moves with the movement of the patient. Further, a nonwoven layer 146 can also be included. An upper surface 148 of the nonwoven layer 146 can be attached to a lower surface 144 of the intermediate layer 138. In addition, a lower surface 150 of the nonwoven layer 146 can be joined to an upper surface 154 of an adhesive layer 152. Further, the adhesive layer 152 can include a lower surface 156 that contains an adhesive that can be used to secure the tube securing device 100 to the patient's skin 160. Additionally, a release liner 158 can be attached to the lower surface 156 of the adhesive layer 152 until such time that the tube securing device 100 is to be placed on the patient's skin 160, at which time the release liner 158 can be removed to expose the lower surface 156 for attachment onto the patient's skin 160. Turning now to FIG. 10, as shown, more than one tube securing device 100 can be used to secure a tube 140 (e.g., a nasoenteric feeding tube) to a patient's skin 160 after insertion of a portion of the tube 140 into an opening 162 (e.g., through a patient's nostril or any other opening through which a tube may be inserted. The tube securing device 100 allows for an easier, more efficient, safer, and less invasive manner for securing such tubes compared to many currently available products and methods.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A tube securing device comprising:
an upper clamp having an upper surface and a lower surface;
a lower clamp having an upper surface and a lower surface; and
a hinge located between the upper clamp and the lower clamp to define a tube insertion region;
wherein a ratio of a length of the upper surface of the upper clamp to a length of the lower surface of the lower clamp ranges from about 0.95 to about 1.20.

2. The tube securing device of claim 1, wherein the length of the upper surface of the upper clamp ranges from about 1.25 millimeters to about 1.7 millimeters.

3. The tube securing device of claim 1, wherein the length of the lower surface of the lower clamp ranges from about 1.2 millimeters to about 1.6 millimeters.

4. The tube securing device of claim 1, wherein the upper surface of the upper clamp and the lower surface of the lower clamp are slidably engaged when the upper clamp is depressed towards the lower clamp to secure a tube within the tube insertion region.

5. The tube securing device of claim 4, wherein the upper surface of the upper clamp and the lower surface of the lower clamp form an angle with each other ranging from about 0° to about 25° when slidably engaged.

6. The tube securing device of claim 1, further comprising a protrusion extending from a base of the tube securing device adjacent the hinge.

7. The tube securing device of claim 1, wherein the tube insertion region has a radius, where a ratio of the radius of the tube insertion region to an outer diameter of a tube inserted into the tube insertion region ranges from about 1:1 to about 1:3.

8. The tube securing device of claim 1, wherein at least one protrusion extends from the lower surface of the upper clamp.

9. The tube securing device of claim 1, wherein the upper surface of the lower clamp includes a first indentation at a first edge, a second indentation at a second edge, and a raised portion defined therebetween.

10. The tube securing device of claim 1, wherein the upper surface of the upper clamp and the lower surface of the upper clamp define a trapezoidal shape where the upper surface and the lower surface meet.

11. The tube securing device of claim 1, further comprising an intermediate layer having an upper surface and a lower surface, wherein the upper surface of the intermediate layer is attached to a lower surface of a base of the tube securing device.

12. The tube securing device of claim 11, further comprising a nonwoven layer having an upper surface and a lower surface, wherein the upper surface of the nonwoven layer is attached to the lower surface of the intermediate layer.

13. The tube securing device of claim 12, further comprising an adhesive layer having an upper surface and a lower surface, wherein the upper surface of the adhesive layer is attached to the lower surface of the nonwoven layer.

14. The tube securing device of claim 13, wherein the lower surface of the adhesive layer includes an adhesive.

15. The tube securing device of claim 13, wherein the lower surface of the adhesive layer is attached to a removable release liner.

16. The tube securing device of claim 1, wherein the tube securing device secures a tube having a size of 6 French, 8 French, 10 French, 12 French, or 14 French.

17. The tube securing device of claim 16, wherein the tube is formed from silicone, polyurethane, polyvinyl chloride, or a combination thereof.

18. The tube securing device of claim 1, wherein the tube securing device is configured to receive a feeding tube.

19. The tube securing device of claim 18, wherein the tube is a nasoenteric feeding tube.
